(19) United States
(12) Patent Application Publication
(10) Pub. No.: US 2022/0088105 A1
(43) Pub. Date: Mar. 24, 2022

(54) METHOD AGAINST CORONAVIRUS INFECTION WITH WATER-EXTRACTED PRODUCT OF PERILLA FRUTESCENS

(71) Applicants: Chang Gung University, Taoyuan City (TW); National Defense Medical Center, Taipei City (TW)

(72) Inventors: Jim-Tong Horng, New Taipei City (TW); Wen-Fang Tang, Taoyuan City (TW); Hui-Ping Tsai, Taipei City (TW); Tien-Yao Chang, Taipei City (TW); Po-Shiuan Hsieh, Taipei City (TW); Yu-Hsiu Chang, Taipei City (TW); Chung-Fan Hsieh, Taoyuan City (TW); Chia-Yi Lin, Taoyuan City (TW); Guan-Hua Lin, Taoyuan City (TW); Yu-Li Chen, Taoyuan City (TW)

(21) Appl. No.: 17/217,561

(22) Filed: Mar. 30, 2021

(30) Foreign Application Priority Data
Sep. 21, 2020 (TW) 109132586

(51) Int. Cl. A61K 36/535; A61K 9/00; A61P 31/14

(52) U.S. Cl. CPC A61K 36/535; A61K 2236/331; A61P 31/14; A61K 9/0053

(57) ABSTRACT

Disclosed herein is a method against coronavirus infection, which includes administering to a subject in need thereof a composition containing a water-extracted product of Perilla frutescens.

### CROSS-REFERENCE TO RELATED APPLICATION

This application claims priority to Taiwanese Invention Patent Application No. 109132586, filed on Sep. 21, 2020.

### FIELD

The present disclosure relates to a method against coronavirus infection with a composition containing a water-extracted product of Perilla frutescens.

### BACKGROUND

Perilla frutescens (L.) Britt., commonly known as Perilla or wild sesame seed, is a species of Perilla in the mint family Lamiaceae. Perilla frutescens is an important economic crop and with a long cultivation history in China as well as some other countries in Asia. Except for the edible applications, the plant of Perilla frutescens has been also traditionally used as a medicinal herb in China for thousands years. The leaves, seeds, and stems of Perilla frutescens are recommended by the Chinese Pharmacopeia as three medicinal materials for various therapeutic applications. Perilla frutescens has been used as an important traditional herbal medicine for treating various diseases/disorders including depression, anxiety, tumors, asthma, cough, allergy, intoxication, vomiting, and some intestinal disorders.

Coronaviruses are a group of related RNA viruses that infect a variety of animal species including humans, such as severe acute respiratory syndrome coronavirus (SARS-CoV), middle east respiratory syndrome coronavirus (MERS-CoV), and human coronavirus 229E (HcoV-229E). Coronavirus disease 2019 (COVID-19) is an infectious disease caused by severe acute respiratory syndrome coronavirus 2 (SARS-CoV-2) recently discovered as a new coronavirus. Major symptoms include respiratory symptoms such as fever above 38° C., cough, shortness of breath, and difficulty in breathing. Symptoms such as loss of smell and taste, diarrhea, headache, chills, loss of appetite, general malaise, and impaired consciousness may be observed. At present, an effective curative treatment for COVID-19 has not been established, and symptomatic treatment is the center.

### SUMMARY

Therefore, an object of the present disclosure is to provide a method against coronavirus infection that can alleviate at least one of the drawbacks of the prior art.

The method includes administering to a subject in need thereof a composition containing a water-extracted product of Perilla frutescens.

### BRIEF DESCRIPTION OF THE DRAWINGS

The above and other objects, features and advantages of the present disclosure will become apparent with reference to the following detailed description and the exemplary embodiments taken in conjunction with the accompanying drawings, in which:

FIG. 1 shows the cell viability rate in each group of Example 2, infra.

### DETAILED DESCRIPTION

It is to be understood that, if any prior art publication is referred to herein, such reference does not constitute an admission that the publication forms a part of the common general knowledge in the art, in Taiwan or any other country.

For the purpose of this specification, it will be clearly understood that the word “comprising” means “including but not limited to”, and that the word “comprises” has a corresponding meaning.

Unless defined otherwise, all technical and scientific terms used herein have the meaning commonly understood by a person skilled in the art to which the present disclosure belongs. One skilled in the art will recognize many methods and materials similar or equivalent to those described herein, which could be used in the practice of the present disclosure. Indeed, the present disclosure is in no way limited to the methods and materials described.

The present disclosure provides a method against coronavirus infection, which includes administering to a subject in need thereof a composition containing a water-extracted product of Perilla frutescens.

As used herein, the term “against coronavirus infection” or “anti-coronavirus infection” means prevention of infection by a coronavirus, suppression of coronavirus replication, and/or treatment and/or prevention of infectious diseases caused by a coronavirus.

As used herein, the term “administration” or “administering” means introducing, providing or delivering a pre-determined active ingredient to a subject by any suitable routes to perform its intended function.

As used herein, the term “subject” refers to any animal of interest, such as humans, monkeys, cows, sheep, horses, pigs, goats, dogs, cats, mice, and rats. In certain embodiments, the subject is a human.

According to the present disclosure, the water-extracted product may be obtained from a plant of Perilla frutescens selected from the group consisting of Perilla frutescens (L.) Britton var. Frutescens, Perilla frutescens var crispa, Perilla frutescens var. purpurascens, Perilla frutescens var. hirtella, and combinations thereof.

According to the present disclosure, the preparation processes and conditions of the water-extracted product of Perilla frutescens are within the expertise and routine skills of those skilled in the art (for example, see Linghua Meng et al. (2009), Molecules, 14, 133-140; and Toshiaki Makinol et al. (2003), Phytother. Res., 17, 240-243).

In certain embodiments, the water-extracted product of Perilla frutescens is prepared by a process including the steps of:

a) admixing a plant material of a plant of Perilla frutescens with water, followed by conducting a heating treatment, such that a mixture is obtained; and

b) removing impurities from the mixture obtained in step (a), followed by collecting an aqueous solution thus formed to obtain the water-extracted product of Perilla frutescens.

According to the present disclosure, examples of the plant material may include, but are not limited to, the flower, stem, leaf, seed, fruit, or fruit peel of the plant of Perilla frutescens, and the entirety of the plant of Perilla frutescens.

According to the present disclosure, the heating treatment is conducted at a temperature ranging from 37° C. to 100° C.

According to the present disclosure, the heating treatment is conducted for a period ranging from 1 hour to 12 hours.

According to the present disclosure, the coronavirus infection may be caused by a coronavirus selected from the group consisting of severe acute respiratory syndrome coronavirus (SARS-CoV), severe acute respiratory syndrome coronavirus 2 (SARS-CoV-2), middle east respiratory syndrome coronavirus (MERS-CoV), human coronavirus 229E (HcoV-229E), and combinations thereof.

According to the present disclosure, the composition containing the water-extracted product of Perilla frutescens may be a pharmaceutical composition that could be formulated into a suitable dosage form for parenteral or oral administration using technology well known to those skilled in the art. The suitable dosage form includes, but is not limited to, injections (e.g., sterile aqueous solutions or dispersions), sterile powder, tablets, troches, lozenges, capsules, dispersible powder, granule, solutions, suspensions, emulsions, syrup, elixirs, slurry, and the like.

According to the present disclosure, the pharmaceutical composition may be administered by parenteral routes selected from the group consisting of intraperitoneal injection, intrapleural injection, intramuscular injection, intravenous injection, intraarterial injection, intraarticular injection, intrasynovial injection, intrathecal injection, intracranial injection and sublingual administration.

The pharmaceutical composition may further include a pharmaceutically acceptable carrier widely employed in the art of drug-manufacturing. For instance, the pharmaceutically acceptable carrier may include one or more of the following agents: solvents, buffers, emulsifiers, suspending agents, decomposers, disintegrating agents, dispersing agents, binding agents, excipients, stabilizing agents, chelating agents, diluents, gelling agents, preservatives, fillers, wetting agents, lubricants, absorption delaying agents, liposomes, and the like. The choice and amount of the aforesaid agents are within the expertise and routine skills of those skilled in the art.

The dosage and the frequency of administration of the pharmaceutical composition may vary depending on the following factors: the severity of the disease to be treated, the route of administration, and the weight, age, physical condition and response of the subject to be treated. The daily dosage of the pharmaceutical composition may be administered in a single dose or in several doses.

According to the present disclosure, the composition containing the water-extracted product of Perilla frutescens may be formulated into an external preparation (such as a hand sanitizer or a hand washing agent) suitable for application to the hands or skin using technology well known to those skilled in the art. The external preparation includes, but is not limited to, an emulsion, a soap, a gel, an ointment, a cream, an aerosol, a spray, a lotion, a serum, a paste, a foam, and a drop.

According to the present disclosure, the composition containing the water-extracted product of Perilla frutescens is easy to apply, low in toxicity, environmentally friendly, and not bioaccumulative, and thus can be used as an environmental disinfectant (such as a surface cleaner, a detergent, and a sterilant).

According to the present disclosure, the pharmaceutical composition may further include remdesivir serving as a synergistic antiviral agent.

The disclosure will be further described by way of the following examples. However, it should be understood that the following examples are solely intended for the purpose of illustration and should not be construed as limiting the disclosure in practice.

### EXAMPLES

### Example 1. Preparation of Water-Extracted Product of Perilla frutescens

5 g of lyophilized powder of Perilla frutescens (which was obtained from the entirety of the plant of Perilla frutescens) was dissolved in 50 mL of sterile ddH₂O, followed by heating in a water bath incubator (37° C.) for 12 hours. The resultant mixture was subsequently filtered using a filter with a porosity of 0.22 μm, so as to obtain a filtrate. The filtrate thus obtained is referred to as “the water-extracted product of Perilla frutescens” hereinafter. Aliquots of the filtrate were dispensed into microcentrifuge tubes and stored at −80° C. for subsequent use.

### Example 2. Evaluation for the Effect of Water-Extracted Product of Perilla frutescens Against Severe Acute Respiratory Syndrome Coronavirus 2 (SARS-CoV-2)

### Experimental Materials:

A. Preparation of Tested Solution of Water-Extracted Product of Perilla frutescens

A suitable amount of the water-extracted product of Perilla frutescens obtained in Example 1 was dissolved in Dulbecco's Modified Eagle's Medium (DMEM)(Cat. No. 12000-061, Gibco) supplemented with 2% fetal bovine serum (FBS)(Cat. No. 26140-079, Gibco), which is referred to as “E2 medium” hereinafter, so as to prepare a stock solution of the water-extracted product of Perilla frutescens having a concentration of 5 mg/mL.

Afterwards, the resultant stock solution was subjected to a two-fold serial dilution using E2 medium, so as to obtain eight tested solutions having different concentrations (i.e., 2.4 μg/mL, 4.9 μg/mL, 9.8 μg/mL, 19.5 μg/mL, 39.1 μg/mL, 78.1 μg/mL, 156.3 μg/mL, and 312.5 μg/mL).

B. Source and Cultivation of Vero E6 Cells

African green monkey kidney (Vero E6) cells were obtained from the Chang Gung Medical Foundation, the Linkou Chang Gung Memorial Hospital (Taiwan). The Vero E6 cells were grown in a 10-cm Petri dish containing DMEM (Cat. No. 12000-061, Gibco) supplemented with 10% FBS (Cat. No. 26140-079, Gibco). The Vero E6 cells were cultivated in an incubator with culture conditions set at 37° C. and 5% CO₂. Medium change was performed every two to three days. Cell passage was performed when the cultured cells reached 80%-90% of confluence.

C. SARS-CoV-2 Solution

A SARS-CoV-2 solution (with a virus amount of 5.73×10⁶ pfu/mL) was provided by the Chang Gung Medical Foundation, the Linkou Chang Gung Memorial Hospital (Taiwan). The SARS-CoV-2 solution was stored in a freezer at −80° C. for further experiment.

### Experimental Procedures:

The Vero E6 cells were divided into 10 groups, including one normal control group, one pathological control group, and eight experimental groups (i.e., experimental groups 1 to 8). Each group of the Vero E6 cells was incubated in a respective well of a 96-well culture plate containing 100 μL of E2 medium at 2×10⁴ cells/well, followed by cultivation in an incubator (37° C., 5% CO₂) for 24 hours. Afterwards, the culture medium in each well was removed, the cells of each of the experimental groups 1 to 8 were added with 50 μL of a respective one of the eight tested solutions prepared in section A, followed by being treated with 150 μL of the SARS-CoV-2 solution described in section C.

In addition, the cells of the pathological control group were added with 50 μL of E2 medium, followed by being treated with 150 μL of the SARS-CoV-2 solution prepared in section C. The cells of the normal control group were added with 200 μL of E2 medium, and were not treated with the SARS-CoV-2 solution prepared in section C.

Each group was cultivated in an incubator (37° C., 5% CO₂) for 96 hours. The liquid in each well was removed, followed by adding 50 μL of 3-[4,5-dimethylthiazol-2-yl]-2,5-diphenyltetrazoli-um bromide (MTT). After cultivation in an incubator (37° C., 5% CO₂) for 2 hours, the respective resultant cell culture was added with 150 μL of dimethyl sulfoxide (DMSO), followed by subjecting the mixture thus obtained to determination of absorbance at a wavelength of 570 nm by an ELISA reader.

The cell viability rate (%) was calculated using the following Equation (I):

A=(B/C)×100 (I)

where A=cell viability rate (%)

B=OD₅₇₀ value of respective group

C=OD₅₇₀ value of normal control group

In addition, the 50% effective concentration (EC₅₀) was determined from the linear portion of the plotted dose-response curve by calculating the concentration of active ingredient that reduced absorbance in the treated cells, as compared to the pathological control cells, by 50% (n=3). The experimental data are expressed as mean±SD (standard deviation).

### Results:

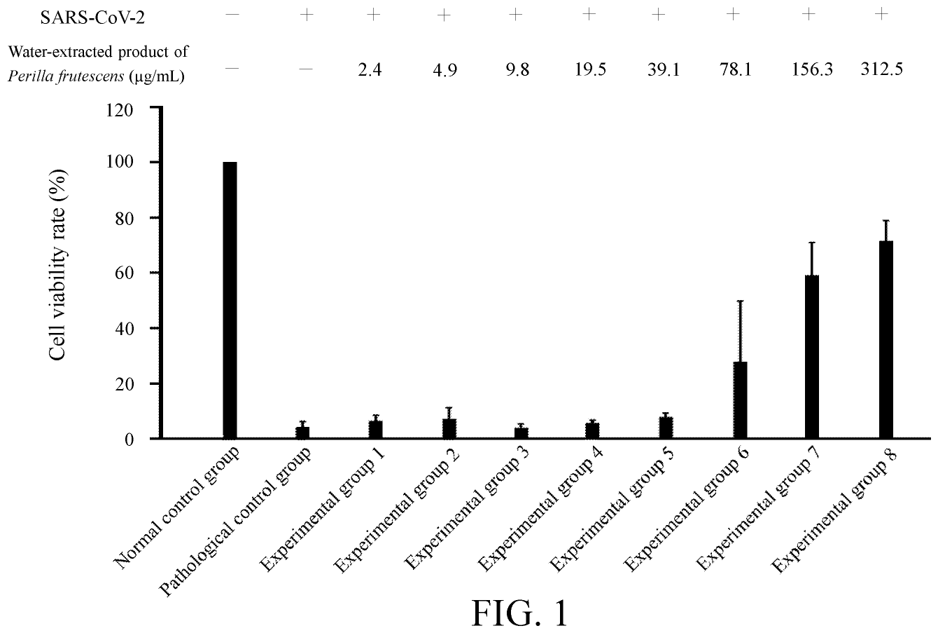

FIG. 1 shows the cell viability rate of each group. It can be seen from FIG. 1 that the cell viability rates determined in the experimental groups 1 to 8 were higher than that determined in the pathological control group, and the water-extracted product of Perilla frutescens exhibited dose-related antiviral effect. In addition, the EC₅₀ value of the water-extracted product of Perilla frutescens is 0.12±0.06 mg/mL.

Summarizing the test results above, it is clear that the water-extracted product of Perilla frutescens can act effectively against coronavirus (such as SARS-CoV-2) infection.

All patents and references cited in this specification are incorporated herein in their entirety as reference. Where there is conflict, the descriptions in this case, including the definitions, shall prevail.

While the disclosure has been described in connection with what are considered the exemplary embodiments, it is understood that this disclosure is not limited to the disclosed embodiments but is intended to cover various arrangements included within the spirit and scope of the broadest interpretation so as to encompass all such modifications and equivalent arrangements.

What is claimed is:

1. A method against coronavirus infection, comprising administering to a subject in need thereof a composition containing a water-extracted product of Perilla frutescens.

2. The method according to claim 1, wherein the coronavirus infection is caused by a coronavirus selected from the group consisting of severe acute respiratory syndrome coronavirus (SARS-CoV), severe acute respiratory syndrome coronavirus 2 (SARS-CoV-2), middle east respiratory syndrome coronavirus (MERS-CoV), human coronavirus 229E (HcoV-229E), and combinations thereof.

3. The method according to claim 1, wherein the composition is a pharmaceutical composition.

4. The method according to claim 3, wherein the pharmaceutical composition is in a dosage form for oral administration.

5. The method according to claim 3, wherein the pharmaceutical composition is in a dosage form for parenteral administration.
